(19) United States
(12) Patent Application Publication
(10) Pub. No.: US 2008/0243157 A1
(43) Pub. Date: Oct. 2, 2008

(54) Applicator For Water-Jet Surgery

(75) Inventors: Ralf Klein, Rottenburg (DE); Jochen Queck, Tubingen (DE); Klaus Fischer, Nagold (DE)

Correspondence Address: DICKSTEIN SHAPIRO LLP, 1825 EYE STREET NW, Washington, DC, 20006-5403, US

(73) Assignee: ERBE Elektromedizin GmbH, Tubingen (DE)

(21) Appl. No.: 11/568,279

(22) PCT Filed: Apr. 15, 2005
(86) PCT No.: PCT/EP05/04045

§ 371 Date: Oct. 25, 2006

(30) Foreign Application Priority Data
Apr. 28, 2004 (DE) 10 2004 020 855.7

(51) Int. Cl. A61B 17/3203

(52) U.S. Cl. 606/167

(57) ABSTRACT

Customary applicators for water-jet surgery comprise a handle, a pressure conduit to supply a working fluid from a pressure source and a nozzle at the end of the pressure conduit to generate a fluid jet with specified properties. It is now proposed to provide a set of molded parts that differ from one another in size and/or shape, that can be detachably, individually connected to the handle to form guide devices to guide the elastically deformable pressure conduit from the handle to the nozzle along a course predetermined by the relevant molded part.

### CROSS-REFERENCE TO RELATED APPLICATIONS

This application is a Section 371 of International Application No. PCT/EP2005/004045, filed Apr. 15, 2005, which was published in the German language on Nov. 10, 2005, under International Publication No. WO 2005/104965 A1 and the disclosure of which is incorporated herein by reference.

### BACKGROUND OF THE INVENTION

The invention relates to an applicator for water-jet surgery.

For the dissection of parenchymal tissue it is becoming increasingly common to employ water-jet surgery. An appliance for this purpose is known, for example, from the document U.S. Pat. No. 6,216,573; it comprises a hand-held instrument in which a distally disposed orifice is connected by way of a coupling to a pressure-resistant delivery tube through which a working fluid, in particular a Ringer solution, can be conducted from a pressure source with a very high pressure. Because of the extremely high working pressure (up to several hundred bar), coupling of this appliance is extremely problematic.

Other appliances for water-jet surgery are known in which the pressure conduit extends from the pressure source to the outlet nozzle, so that no separate coupling is needed. In view of the fact that it is impossible to sterilize the pressure tube after it has been used, operations with such an appliance are very expensive, especially when during the operation appliances with different applicators must be used in order to treat sites that are not readily accessible.

### BRIEF SUMMARY OF THE INVENTION

It is the object of the present invention to provide an applicator for water-jet surgery such that, in a simple and economical manner, the number of ways in which the applicator can be employed is increased.

According to the present invention there is provided an applicator for water-jet surgery that comprises a handle, a pressure conduit for supplying a working fluid from a pressure source, a nozzle at the end of the pressure conduit to produce a jet of fluid, and a set of molded parts that differ from one another in size and/or shape and that can be detachably connected, individually to the handle, in which position each forms a guide device to guide the elastically deformable pressure conduit from the handle to the nozzle along a course predetermined by the respective molded part.

Hence, in an applicator according to the invention during an operation the pressure tube is continuously connected to the pressure source and only the molded part is exchanged, to reconfigure the applicator so that its shape is suitable for the momentary operation step; this alteration is performed simply by changing to another molded part during the operation, while the pressure tube is retained.

The guide device is preferably constructed such that the pressure conduit attached to the handle can be removed from the guide device while the molded part is being exchanged and can then be inserted again. This makes it easy to exchange the guide device.

In one embodiment of the invention the molded parts are constructed in tubular form, so that the guide devices consist of the lumen of these tubes. This embodiment is particularly easy to manufacture.

Preferably suction devices are provided, to aspirate working liquid and also blood or other body liquids. This is especially simple when the molded parts are tubular and the lumen can be attached to a suction device.

The set of molded parts preferably comprises at least one straight molded part and one molded part that is curved or bent at an angle. With these two embodiments, most applications can be performed. It is advantageous for these molded parts to be available in various sizes, to meet the demands of all operational conditions.

In an especially preferred embodiment the molded part comprises at least at its distal end, directed away from the handle, electrode devices that can be connected to a HF surgical generator for the coagulation of tissue. With this embodiment, therefore, another substantial problem encountered in such operations is solved, as follows. In order to expose and take care of vessels or bile ducts (in the case of the liver) ligatures, clips or high-frequency current have so far been used, and whenever this occurs it has been necessary to exchange instruments. The time thereby consumed is extraordinarily stressful, for the patient as well as for the person doing the operation. This applicator design in accordance with the invention can eliminate the need for such an instrument exchange.

Electrode devices of this kind can be designed in various ways. For instance, it is possible to allow a coagulation current to flow by making direct contact between the electrode and the tissue. In another preferred embodiment the molded part comprises a device, in particular a tubular probe, to supply a noble gas, as well as an electrode preferably disposed within the probe to supply a HF current; thus it forms a noble-gas-plasma coagulation device, such as is known per se.

The molded part further preferably comprises a conduit extending to the nozzle in order to supply or remove fluids, so that it is possible to rinse the operation area and clear it by suction. The suction conduit preferably comprises a suction opening, which preferably is deflected in a direction determined by the nozzle. As a result, suction and application of the water jet can occur simultaneously, without disrupting the jet.

Preferably the pressure conduit with nozzle, in some cases together with the handle, is intended to be used only once, whereas the molded part is designed to be sterilizable. Thus the operation costs can be kept low.

The molded part and/or the pressure conduit preferably comprise adjustment devices, designed to enable the nozzle to be placed in a specific position relative to the molded part. In this way the attainable precision of the operation is increased.

In this embodiment the adjustment devices can comprise a pressing device with which to push the pressure conduit in the direction of the fluid jet so that it comes into contact with a stop formed on the molded part. By this means not only the direction but also the “end position” of the nozzle is specified relative to the handle. The pressure conduit can comprise a deformable end section containing the nozzle, which can be connected by way of a coupling to a supply section that leads to the pressure source. In this way, especially when the molded parts are of different lengths, a better match between pressure tube and molded part can be achieved. This also makes it possible for the end section to be exchanged together with the molded part, which in some circumstances can simplify the exchange of molded parts.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is a side elevation of a handle with pressure conduit of an applicator for water jet surgery,

FIG. 2 is a side elevation of a first embodiment of a molded part for use with the handle shown in FIG. 1,

FIG. 3 shows a partial longitudinal section of the connection site between the molded part and the handle shown in FIGS. 2 and 1 respectively,

FIG. 4-1 to FIG. 4-5 are diagrams showing various preferred shapes of molded parts,

FIG. 5 is a side elevation showing a handle with a molded part attached,

FIGS. 6 to 8 are sections through the region VI-VIII of FIG. 5 in three different embodiments of molded part,

FIG. 9 is a partial longitudinal section through a handle end on the supply side,

FIG. 10 is a view similar to that of FIG. 9 and showing another embodiment of a handle,

FIG. 11 is a view similar to that of FIG. 1 but showing another embodiment of handle,

FIG. 12 is a view similar to that of FIG. 2 but showing another embodiment of molded part for use with the handle shown in FIG. 11,

FIG. 13 is an enlarged sectional drawing of the coupling region between the handle and the molded part shown in FIGS. 11 and 12 respectively, and

FIG. 14 is a schematic drawing of another molded part with HF-coagulation devices.

### DETAILED DESCRIPTION OF THE INVENTION

In the following description, the same reference numerals are used for identical parts or parts with identical actions.

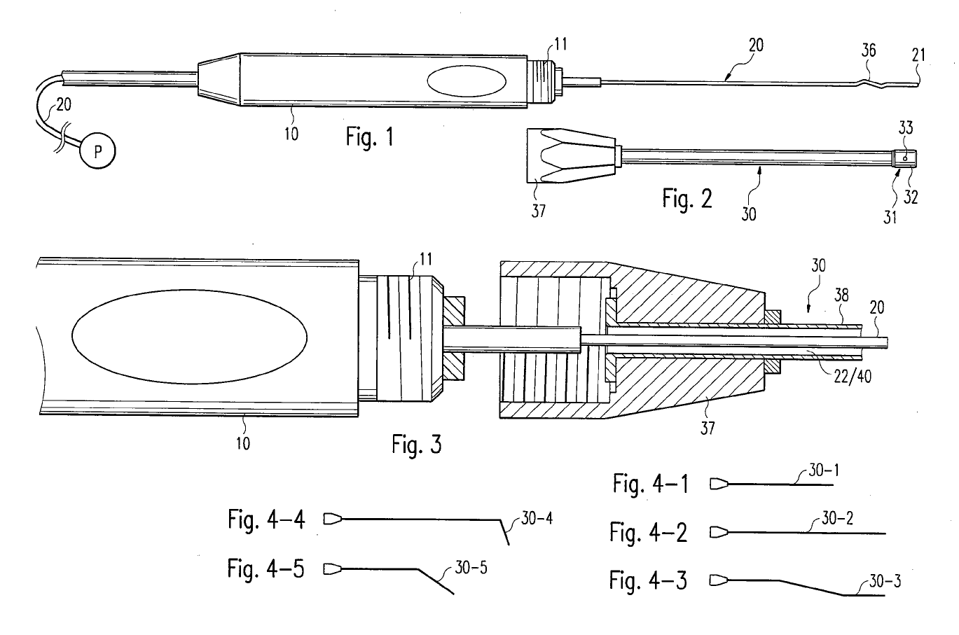

As can be seen in FIGS. 1 to 3, the applicator comprises a handle 10 through which a pressure conduit 20 passes from a pressure source P to a nozzle 21. So that a molded part 30 can be attached, the distal end of the handle 10 is provided with a screw thread 11 onto which a correspondingly configured thread in a coupling cap 37 can be rotated. Although the coupling cap 37 is rotatably seated on a tube 38, it is fixed in position in the longitudinal direction. The pressure conduit 20 passes through the lumen 40 of the tube 38 and at its distal end, in the embodiment shown here, comprises a centering spiral 36. This centering spiral or wave is as shown in FIGS. 6 and 7—dimensioned such that the end of the pressure conduit 20 is firmly held within the lumen 40 of the tube 38 and the nozzle 21 can be positioned substantially in the center of the tube 38, just ahead of its end.

At the distal end 31 of the tube 38, i.e. of the molded part 30, there is an end piece 32 with laterally positioned suction openings 33. In this case the lumen 40 simultaneously constitutes a conduit 22 that can be connected to a suction device.

As shown in FIGS. 4-1 to 4-5, the molded parts 30 can be constructed with different lengths (30-1, 30-2) and can be bent into various shapes (30-3 to 30-5), and in the latter case can also vary in length. These shapes and lengths correspond to the requirements associated with the various operations.

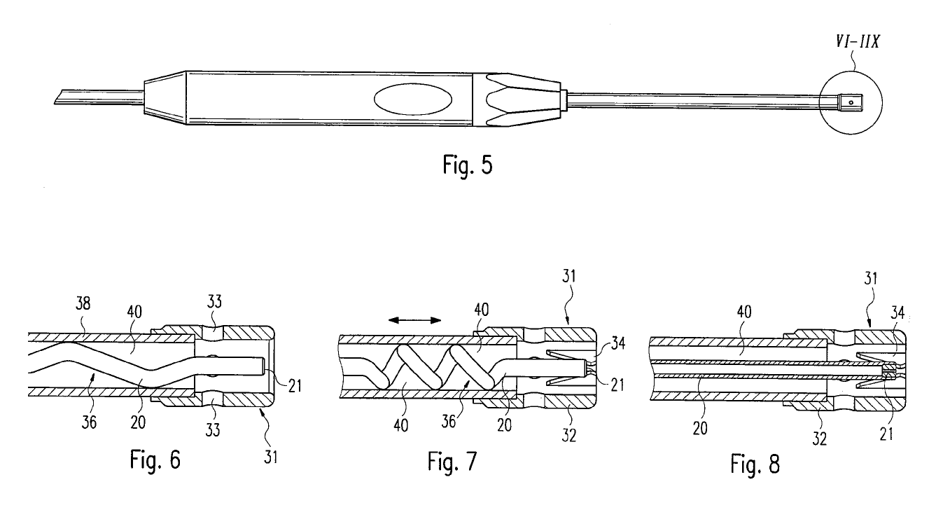

During use, the pressure tube 20 is connected to the pressure source. Then the operator selects the particular molded part 30-1 to 30-5 that is to be used for the first steps in the operation and attaches this molded part 30 to the handle 10, by inserting the pressure tube 20 into the molded part 30, through the lumen 40 of the tube 38, and screwing the coupling cap 37 onto the thread 11 of the handle 10 so that it is fixed in position, with the nozzle 21 at the distal end 31 of the molded part 30. If the operator wants to employ another molded part, for instance one that is bent as shown in FIG. 4-5, then he unscrews the previously used molded part 30-1 and replaces it by a molded part 30-5 by carrying out the steps previously described. After the end of the operation the molded parts 30 can be sterilized and made available for a new operation; the pressure tube 20, in some cases together with the handle, is not used again, as it cannot be sterilized because of the small diameter of its lumen and nozzle.

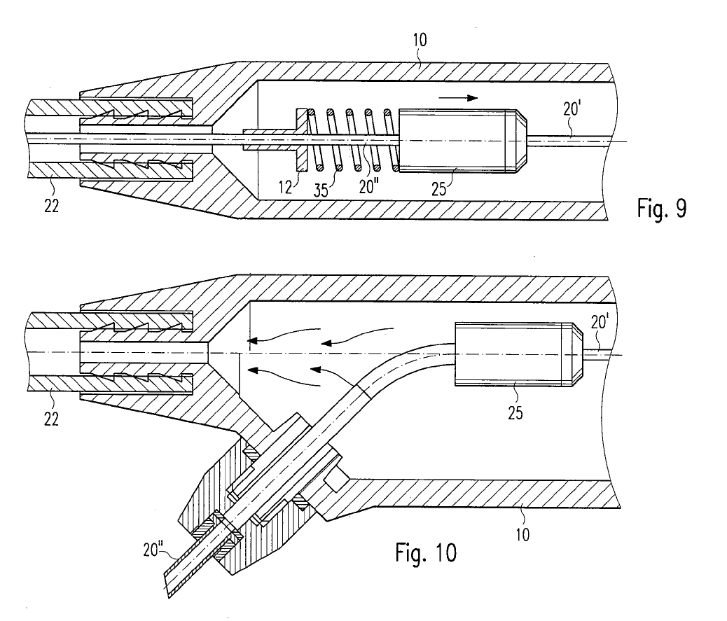

So that the nozzle 21 can be centered within the distal end 31 of the molded part 30, it is possible, as shown in FIGS. 7 and 8, to mount an adjustment stop 34 within the end piece 32 in such a way that during assembly of handle 10 and molded part 30, the end of the pressure conduit 20 with the nozzle 21 is inserted into the adjustment stop 34, which preferably has a conical structure, and is fixed there in a final position; thus the situation of the nozzle 21 is precisely specified, in both the longitudinal direction of the tube 38 and the radial direction of the lumen 40. Differences in length can be compensated either by the centering spiral 36 or—as shown in FIG. 9—by means of a spring 35 that bears against a spring seating 12 in the handle and pushes an end section 20′ of the pressure conduit 20 toward the nozzle, by way of a holder 25 that is fixed to the pressure conduit 20.

The holder 25 can also be simultaneously constructed as a coupling, as shown in FIG. 10, by way of which a thinner, distal end section 20′ of the pressure conduit 20 is connected to a thicker supply section 20″.

The embodiment of the handle according to FIG. 10 also differs from that in FIG. 9 in that the suction conduit 22 does not surround the pressure tube 20 coaxially, but rather is designed as a separate conduit, while the pressure tube 20, or its supply section 20″, emerges through another (sealed) opening in the handle 10.

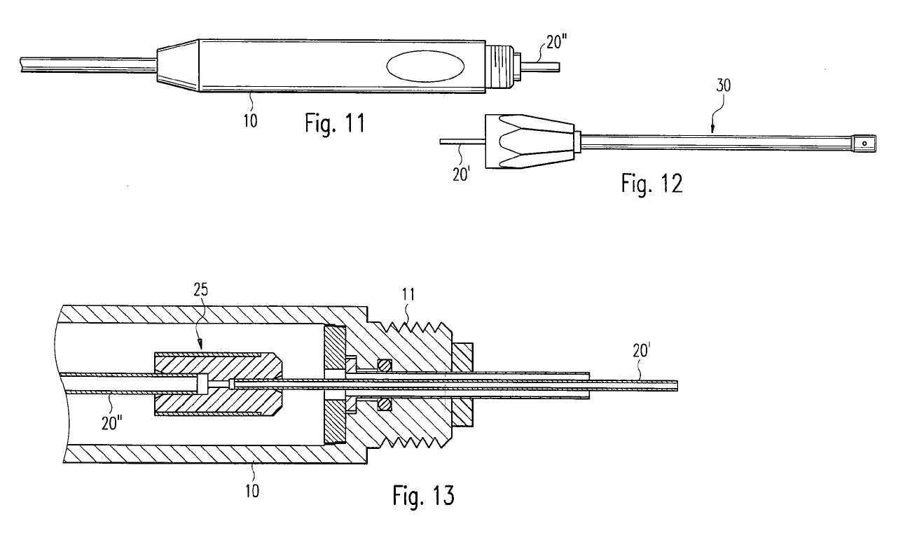

The embodiment of the invention shown in FIGS. 11 to 13 differs from that previously described in that when a molded part 30 is being exchanged, the end section 20′ of the pressure conduit is exchanged along with it. For this purpose a coupling 25 is positioned in the handle 10 in such a way that as the molded part 30 is being put into place and screwed on, the end of the end section 20′ that is opposite the nozzle 21 is inserted into the coupling 25 fixed within the handle 10 and, because of the elastic structure of the coupling 25, is then connected in a pressure-proof manner to the supply section 20″ of the pressure conduit 20.

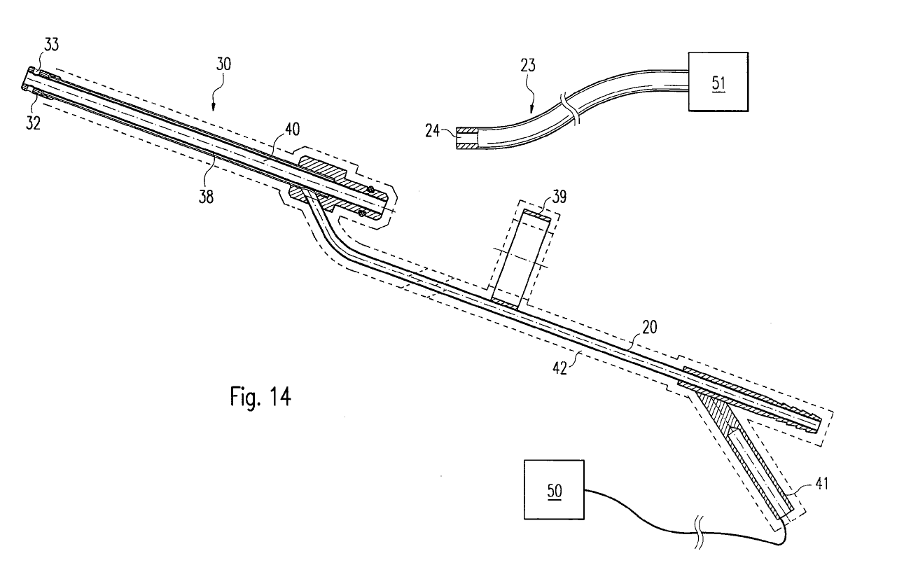

The embodiment of the invention shown in FIG. 14 comprises a molded part 30 that makes it possible to coagulate tissue with HF current. The handle 10 from which the pressure tube 20 projects is connected, as in the case of the “simple” molded parts 30-1 to 30-4 (although here instead of the threaded connection a simpler plug-in connection is shown), in such a way that the pressure tube 20 is fixed to the nozzle 21 in the end piece 32. In this case the molded part 30 has a holder 39 with which to attach this molded part to the handle.

The molded part 30 additionally comprises a HF connector 41 that can be connected to a HF generator 50. The suction conduit 20 is made of metal and joined to a likewise metallic tube 38. The entire arrangement is covered by an insulating layer 42, which insulates it from the exterior as a whole except for the (metallic) end piece 32, which in this embodiment of the invention serves as a coagulation electrode. Hence with this instrument the operator can cut by means of the water jet and simultaneously, when necessary, coagulate tissue by means of the “electrode” 32.

Alternatively or additionally a probe 23 with interior electrode 24, which are connected to an APC coagulation device 51, can be pushed into the lumen 40 together with the pressure tube 20. The adjustment stop 34 (not shown in this figure) is altered accordingly, and is constructed so that both the probe 23 (which is known per se) and the pressure tube 20 are positioned within the end piece 32 in an exactly reproducible manner.

The features of the handle and the molded parts described above can be substantially arbitrarily combined with one another, so that a multifunctional instrument is produced.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. Applicator for water-jet surgery, comprising a handle an elastically deformable pressure conduit for the supply of a working fluid from a pressure source, a nozzle located at the end of the pressure conduit and adapted to generate a fluid jet with predetermined properties, a set of molded parts that differ from one another in at least one of size and shape and that can each be detachably connected, individually to the handle in which position each forms a guide device with which to guide the elastically deformable pressure conduit from the handle to the nozzle along a course predetermined by the size and shape of said respective molded part.

2. Applicator according to claim 1, wherein said pressure conduit is fixed to said handle, and said guide devices are each adapted such that, during exchange of the guide devices, the pressure conduit can be detached from one guide device and inserted into another guide device.

3. Applicator according to claim 1, wherein each of said guide devices comprises a tubular molded part defining a lumen.

4. Applicator according to claim 3, wherein the lumen isadapted to be connected to a suction device.

5. Applicator according to claim 1, wherein said set includes at least one straight molded part and one curved or angled molded part.

6. Applicator according to claim 1, wherein at least one of said molded parts comprises, at least at its distal end directed away from the handle (10), an electrode device that is adapted to be connected to a HF surgical generator for the coagulation of tissue.

7. Applicator according to claim 1, wherein at least one of said molded parts comprises a conduit that extends to the nozzle and serves to supply or remove fluids.

8. Applicator according to claim 7, wherein said conduit defines at least one suction opening which is deflected in a direction predetermined by the nozzle.

9. Applicator according to claim 6, wherein at least one said molded part comprises a probe adapted to supply noble gas and an electrode device disposed within the probe and adapted to supply a HF current in order to form a noble-gas-plasma coagulation apparatus.

10. Applicator according to claim 1, wherein said pressure conduit, said nozzle and said handle are intended to be used only once, whereas said molded parts are adapted to be sterilizable.

11. Applicator according to claim 1, wherein at least one of the molded parts and the pressure conduit comprises an adjustment device that enables the nozzle to be placed in a specified position with respect to the molded part.

12. Applicator according to claim 11, wherein the molded part comprises a centering stop and the adjustment device comprises a pressing device adapted to push the pressure conduit in the direction of the fluid jet, against said centering stop formed on the molded part.

13. Applicator according to claim 12, wherein said pressure conduit comprises a deformable end section that contains the nozzle and that is adapted to be connected by way of a coupling to a supply section that leads to the pressure source.

14. Applicator according to claim 13, wherein said end section can be exchanged together with the molded part.
